(19) United States
(12) Patent Application Publication
(10) Pub. No.: US 2024/0299285 A1
(43) Pub. Date: Sep. 12, 2024

(54) PERSONAL CARE COMPOSITION

(71) Applicant: The Procter & Gamble Company, Cincinnati, OH (US)

(72) Inventors: Debora W. Chang, Mason, OH (US); Joshua Mikell Williams, Cincinnati, OH (US)

(21) Appl. No.: 18/599,698

(22) Filed: Mar. 8, 2024

Related U.S. Application Data
(60) Provisional application No. 63/489,023, filed on Mar. 8, 2023.

(51) Int. Cl. A61K 8/88

(52) U.S. Cl. CPC A61K 8/88; A61K 2800/30; A61K 2800/5426

(57) ABSTRACT

An aqueous, sulfate-free personal care composition that provides good cleaning, foaming, lathering, viscosity and stability. The composition includes an acyl taurate surfactant and an N-alkyl acyl taurate surfactant at a weight ratio of acyl taurate to N-alkyl acyl taurate of 1:4 to 50:1. The composition also includes a co-surfactant and water. The N-alkyl acyl taurate surfactant and co-surfactant are specifically tailored to solubilize the acyl taurate surfactant in the composition and thereby deliver the desired composition benefits.

The present disclosure generally relates to a personal care composition comprising an acyl taurate surfactant and an N-alkyl acyl taurate surfactant. More specifically, the present disclosure relates to a personal care compositions comprising a combination of an acyl taurate surfactant, an N-alkyl acyl taurate surfactant and a co-surfactant at a ratio that provides improved solubility and lather.

### BACKGROUND

Human hair becomes soiled due to contact with the surrounding environment and from sebum secreted by the scalp. Soiled hair can have an undesirable feel and/or appearance. As a result, people may clean their hair with a shampoo composition that restores the hair to a clean and attractive appearance. Many conventional shampoos use sulfated surfactants such as sodium lauryl sulfate (SLS) and/or sodium laureth sulfate (SLES) to clean hair. Sulfated surfactants are generally very good at removing oil and other contaminants from hair, but they also have drawbacks. For example, sulfated surfactants are sometimes associated with poor quality hair feel, hair dryness and/or skin dryness after washing. This is commonly referred to as “harshness,” and harsh shampoos generally face poor consumer acceptance.

Sulfate free surfactants, such as isethionate-based surfactants, are sometimes used to avoid some of the perceived harshness of sulfated surfactants. For example, Indian patent application No. 3544/DEL/2015 discloses that sodium cocoyl isethionate (SCI) is the primary sulfate free surfactant of choice for its mildness towards hair and skin. While SCI may provide good cleaning and mildness, it may not be suitable for use in certain liquid personal care compositions because of its relatively poor solubility in water. Compositions that use SCI as the primary detersive surfactant typically require the addition of high levels of co-surfactant to prevent the SCI from precipitating out. In addition, SCI may not be suitable for use at acidic pH (e.g., <pH 6) because it is susceptible to hydrolysis, which results in decreased foaming, cleaning and stability.

Another class of relatively mild surfactants known for use in personal care are N-alkyl acyl taurates such as sodium methyl cocoyl taurate (SMCT) and sodium methyl lauroyl taurate (SMLT). SMCT and SMLT are readily available at commercial scale, exhibit better solubility in water than SCI and do not hydrolyze at acidic pH like some other amino acid-based surfactants (e.g., glycinates, sarcosinates, alaninates and glutamates). SMCT and SMLT are milder than most sulfated surfactant and generally provides good cleaning benefits for skin and hair. However, the methyl group bonded to the amide nitrogen in SMCT and SMLT makes it difficult to build viscosity in an aqueous personal care composition using an inorganic salt, which is typically how viscosity is built in a surfactant-based composition such as shampoos and body washes. Viscosity is important in personal care compositions because compositions with insufficient viscosity can be difficult to dispense and apply in a controlled manner and/or may be perceived as low quality.

Another taurate surfactant that may be used in a personal care composition is an acyl taurate surfactant such as sodium cocoyl taurate (SCT) or sodium lauroyl taurate (SLT). SCT and SLT do not have an alkyl group bonded to the amide nitrogen, and thus does not have the viscosity building drawbacks of their N-alkyl counterparts. However, acyl taurates tend to be less soluble than N-alkyl acyl taurates in water, and they may not be commercially available at the volumes necessary for mass production of personal care products. These drawbacks make acyl taurate surfactants undesirable for use in many personal care products.

Sulfate-free conditioning shampoos (i.e., shampoos that provide a cleansing and conditioning benefit to hair) face additional challenges. Conditioning shampoos typically use a cationic conditioning polymer to form a coacervate with an anionic surfactant system during use, which deposits on hair to provide a conditioning benefit (e.g., casier wet combing and detangling). However, the formation of coacervate ties up a portion of the surfactant within the coacervate and therefore decreases the amount of surfactant available to provide foaming and cleansing. Since non-sulfated surfactants tend to be less effective than sulfated surfactants at foaming and cleansing to begin with, the addition of a cationic polymer just further taxes the surfactant system, resulting in even less foaming and cleansing.

Thus, there is a need for gentle cleansing compositions that provide desirable foam and cleansing properties with naturally derived surfactants, especially in conditioning shampoos.

Accordingly, it would be desirable to provide a personal cleansing composition that includes a sulfate-free surfactant with suitable solubility, stability, foaming and cleansing properties. It would also be desirable to provide a sulfate free personal care composition that includes a cationic polymer and does not compromise on foaming and cleansing. It would further be desirable to provide a personal care composition with sulfate-free surfactants that do not impede viscosity building.

### SUMMARY

Disclosed herein is an aqueous personal care composition comprising an acyl taurate surfactant and an N-alkyl acyl taurate surfactant at a weight ratio of acyl taurate to N-alkyl acyl taurate of about 1:4 to about 50:1. The composition also includes a co-surfactant and water. The N-alkyl acyl taurate surfactant and co-surfactant are specifically tailored to solubilize the acyl taurate surfactant in the composition, which results in a composition with suitable cleaning, foaming, lathering, viscosity and stability. The composition has a consumer-preferred viscosity of about 2000 mPa-s to about 20,000 mPa-s, according to the Rheology method.

### BRIEF DESCRIPTION OF THE DRAWING

FIG. 1 is a chart illustrating the solubility of SMCT, SCT and SCI.

FIG. 2 is a chart illustrating the solubility of an SCT/SMCT mixture and SCI

FIG. 3 is chart illustrating the synergistic lather height characteristics of an SCT/SMCT mixture.

FIGS. 4A-4H illustrate the effect of co-surfactant on acyl taurate solubility.

### DETAILED DESCRIPTION

Recent trends indicate a desire by consumers to replace their sulfated cleansing compositions with milder, sulfate free versions. However, conventional sulfate free personal care compositions may be perceived as poor performing due to, for example, low foam generation. In addition, some sulfate free surfactants interact with other ingredients (e.g., viscosity building inorganic salts and/or cationic conditioning agents) resulting in poor solubility. Surprisingly, it has now been found that certain combinations of acyl taurates and N-alkyl acyl taurates (e.g., methyl acyl taurate) can provide desired foam properties, gentle cleansing, suitable solubility and do not interfere with viscosity building inorganic salts. It has further been found that these combinations of taurates can also be combined with a cationic polymer (e.g., cationic conditioning polymers) with little or no undesirable impact on foam properties.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition, unless specifically stated otherwise, and may be designated as “wt %.” All ratios are weight ratios, unless specifically stated otherwise. All such percentages or weights as they pertain to listed ingredients are based on the active level and, therefore, do not include carriers or by-products that may be included in commercially available materials. The number of significant digits conveys neither a limitation on the indicated amounts nor on the accuracy of the measurements. Unless otherwise indicated, all measurements are understood to be made at approximately 25° C. and at ambient conditions, where “ambient conditions” means conditions under about 1 atmosphere of pressure and at about 50% relative humidity. All ranges are inclusive and combinable. For example, all numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range of plus or minus 20% or less of the stated value (e.g., plus or minus 15% or less, 10% or less, or even 5% or less).

“Apply” or “application,” as used in reference to a composition, means to apply or spread the composition onto a human keratinous surface such as the skin or hair.

“Charge density” (“CD”) means the ratio of positive charges on a polymer to the molecular weight of the polymer.

“Cleansing composition” refers to a personal care composition or product intended for use in cleaning a bodily surface such as skin or hair. Some non-limiting examples of cleansing compositions are shampoos, conditioners, conditioning shampoos, shower gels, liquid hand cleansers, facial cleansers, and the like.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration and food additives.

“Suitable for application to human hair” means that the personal care composition or components thereof, are acceptable for use in contact with human hair and the scalp and skin without undue toxicity, incompatibility, instability, allergic response, and the like.

“Substantially free of” means a composition or ingredient comprises less than 3% of a subject material, by weight of the composition or ingredient (e.g., less than 2%, less than 1% or even less than 0.5%). “Free of” means a composition or ingredient contains 0% of a subject material.

“Sulfated surfactants” means surfactants that contain a sulfate moiety. Some non-limiting examples of sulfated surfactants are sodium lauryl sulfate, sodium laureth sulfate, ammonium lauryl sulfate, and ammonium laureth sulfate. “Sulfate-free surfactant” refers to a surfactant that has no sulfate moieties.

### Personal Care Composition

The sulfate-free personal care compositions herein include a detersive taurate surfactant for cleaning a target bodily surface such as hair and skin. In some instances, the personal care composition may include a co-surfactant, for example, to help solubilize the detersive surfactant or another ingredient in the composition. The taurate surfactant includes an acyl taurate surfactant and an N-alkyl acyl taurate surfactant (e.g., N-methyl, N-ethyl, N-propyl or N-butyl acyl taurate surfactant). The present composition may also include a cationic conditioning polymer to aid in the appearance and/or feel of hair. It has surprisingly been found that when a suitable acyl taurate surfactant is combined with an N-alkyl acyl taurate surfactant, improved lathering is observed, even in the presence of a cationic polymer. In addition, the combination of acyl taurate surfactant and N-alkyl acyl taurate surfactant exhibits unexpected solubility and stability benefits.

The personal care compositions herein may be provided in various product forms such as solutions, suspensions, shampoos, conditioners, lotions, creams, gels, toners, sticks, sprays, aerosols, ointments, cleansing liquid washes, solid bars, pastes, foams, mousses, shaving creams, wipes, strips, patches, hydrogels, film-forming products, facial and skin masks (with and without insoluble sheet), and the like. The composition form may follow from the particular dermatologically acceptable carrier chosen. In some aspects, the personal care compositions described herein may include a dispersed gel network phase that provides a milder, but effective, conditioning benefit to hair in combination with a detersive taurate surfactant.

Liquid personal care compositions herein, such as shampoos, conditioners, and body washes may have a viscosity of 2,000 mPa-s to 20,000 mPa-s (e.g., 2,500-15,000 mPa-s, 3,000-10,000 mPa-s or 3,500-9,000 mPa-s) according to the Rheology method described in more detail below. It is believed that viscosities in this range are generally preferred by users of liquid personal care compositions.

In some aspects, the compositions herein may contain an inorganic salt thickener such as sodium chloride, potassium chloride, sodium sulfate, ammonium chloride, sodium bromide, combinations of these and the like. In some aspects, the inorganic salt may be present at 0-2%, (e.g., 0.05-1% or 0.1-0.5%). In some sulfate-free cleansing compositions, inorganic salt can introduce instability to the composition by aiding in the formation of a coacervate between anionic surfactants and cationic polymers which may be present. The coacervate typically has a gel-like consistency which can precipitate, and it can impact the rheological and performance properties of the composition as well as the consumer-perceived quality of the product. Thus, it can be important to specifically tailor the amount of inorganic salt in the composition formulation. Of course, it is to be appreciated that when the cleansing composition is used as intended, it will form a coacervate upon dilution to provide the desired conditioning benefit.

### Taurate Surfactant

The sulfate-free surfactant system of the personal care compositions herein includes a combination of an acyl taurate surfactant and an N-alkyl acyl taurate to provide foaming and cleaning properties to the personal care compositions herein. Such surfactants are sometimes referred to as detersive surfactants. Detersive surfactants facilitate cleaning due to their amphiphilic nature, which allows the surfactants to break up, and form micelles around, oil and other contaminants on the hair. The “entrapped” contaminant can then be rinsed off more easily with water.

The composition may include 1-20% of taurate surfactant (e.g., 2-15%, 3-12%, or 4-10%). The taurate surfactant may include a weight ratio of acyl taurate to N-alkyl acyl taurate of 1:4 to 50:1 (e.g., 1:4, 1:3, 1:2, 1:1, 2:1, 3:1, 4:1 5:1, 6:1, 7:1, 8:1, 9:1, 10:1, 11:1, 12:1, 13:1, 15:1 20:1, 25:1, 30:1, 35:1, 40:1 or 45:1). It can be important not to include too much N-alkyl acyl taurate, as it can interfere with the ability to build viscosity in the composition, for example, using inorganic salt such as sodium chloride and/or potassium chloride. Compositions with insufficient viscosity (i.e., “runny”) may be viewed as poor quality by a consumer.

Acyl taurate surfactants that may be suitable for use herein are generally described by Formula I illustrated below.

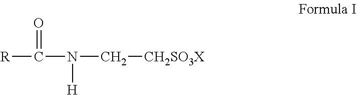

Where: R is an alkyl group with 5 to 23 carbon atoms (e.g., 7-21, 7-17, 7-15, 7-13, 11-17, 11-15, 11-13 or even 11 carbon atoms) and X is a suitable counterion (e.g., sodium, potassium, magnesium, ammonium or triethanolamine).

Some nonlimiting examples of acyl taurates are capric ester taurate, cocoyl taurate, lauroyl taurate, myristoyl taurate, caproyl taurate, oleoyl taurate, capryloyl taurate, palmitoyl taurate, stearoyl taurate, linoleoyl taurate, salts of these and combinations thereof.

The N-alkyl acyl taurate surfactants herein are generally described by formula II illustrated below.

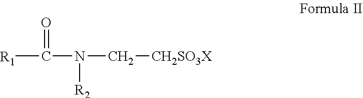

Where: R₁ is an alkyl group with 5 to 23 carbon atoms (e.g., 7-21, 7-17, 7-15, 7-13, 11-17, 11-15, 11-13 or even 11 carbon atoms) and X is a suitable counterion (e.g., sodium, potassium, magnesium, ammonium or triethanolamine) and R₂ is an alkyl group with 1 to 4 carbon atoms. Some nonlimiting examples of N-alkyl acyl taurates that may be suitable for use herein include methyl capric ester taurate, methyl cocoyl taurate, methyl lauroyl taurate, methyl myristoyl taurate, methyl caproyl taurate, methyl oleoyl taurate, methyl capryloyl taurate, methyl palmitoyl taurate, methyl stearoyl taurate, methyl linoleoyl taurate, salts of these and combinations thereof.

It is to be appreciated that the taurate surfactants described herein are typically not single compounds, as suggested by their general formula (I) or (II), but rather a mixture of several homologs having varied chain lengths and molecular weights. Additionally, the taurate surfactants herein may be either saturated or unsaturated.

### Co-Surfactants

The personal care composition herein may include a co-surfactant selected from anionic surfactants, amphoteric surfactants, zwitterionic surfactants, non-ionic surfactants and combinations of these. Some non-limiting examples of anionic surfactants include non-taurate, non-sulfate anionic surfactants such as isethionates, carboxylates, sulfonates (e.g., alpha olefin sulfonates, linear alkylbenzene sulfonates, alkyl glyceryl sulfonates, sodium laurylglucosides hydroxypropylsulfonate), branched alkyl sulfates, sulfosuccinates, sulfoacetates, sulfolaurates, amino acid-based surfactants (e.g., glycinates, sarcosinates, alaninates, glutamates), lactate- and lactylate-based surfactants (e.g., sodium lauroyl lactate and sodium lauroyl lactalyte), phosphate ester surfactants and combinations thereof.

Some non-limiting examples of amphoteric and/or zwitterionic surfactants include derivatives of aliphatic secondary and tertiary amines in which one of the aliphatic substituents contains from 8 to 18 carbon atoms and one aliphatic substituent contains an anionic group such as a carboxy, sulfonate, phosphate, or phosphonate group. Zwitterionic surfactants are surfactants whose polar functional group has two permanent charges that do not change with changing pH. Amphoteric surfactants have polar functional groups whose charge depends on the pH of the solution and can exhibit different charges as the pH changes from acid to neutral to basic, ranging from cationic to zwitterionic and potentially even to anionic. Some non-limiting examples of zwitterionic surfactants include amidosulfobetaines, hydroxysultaines, amidopropyl hydroxysultaines, and combinations thereof. Some non-limiting examples of amphoteric surfactants include amphoacetates, amphodiacetates, betaines, amidobetaines (e.g., cocamidopropyl betaine and lauramidopropyl betaine), propionates, hydroxysultaines, and combinations thereof.

Some non-limiting examples of non-ionic surfactants include glyceryl esters of alkanoic acids, polyglyceryl esters of alkanoic acids, propylene glycol esters of alkanoic acids, sorbitol esters of alkanoic acids, alkanolamides, alkoxylated amides, alkyl glycosides, alkyl polyglucosides acyl glucamides, amine oxides and combinations thereof. Some particularly suitable examples of non-ionic surfactants include cocamide, cocamide MEA, PPG-2 cocamide, PPG-2 hydroxyethyl cocamide, PPG-2 hydroxyethyl isostearamide, lauroyl/myristoyl methyl glucamide, capryloyl/caproyl methyl glucamide, cocoyl methyl glucamide, decyl glucoside, coco-glucoside, lauryl glucoside, lauramine oxide, cocamine oxide and combinations thereof.

More specific examples of the optional co-surfactants described above are disclosed in US 2019/0105246, US 2018/0098923, U.S. Pat. No. 9,271,908, WO 2020/016097, and McCutcheon's Emulsifiers and Detergents, 2019, MC Publishing Co.

The cosurfactant may be present in the personal care compositions at 1% to 15% (e.g., 2-10%, 3-9%, 4-8%, or even 5-7%). The amount of co-surfactant in the composition can be important and should be tailored to balance solubility and/or viscosity building with cleaning and/or conditioning benefit. For example, too much amphoteric co-surfactant can make the surfactant system less salt tolerant and may impede the ability of the surfactant system to form a suitable coacervate upon dilution with water. This can be especially problematic when the composition contains a cationic polymer because the lowered salt tolerance of the surfactant system may cause the cationic polymer to precipitate out. In some embodiments, the composition may include a weight ratio of total taurate surfactant to co-surfactant of 12:1 to 3:10 (6:1 to 3:10, 4:1 to 1:3, or even 2:1 to 1:2).

### Cationic Polymer

The personal care compositions herein may include 0.05-3% of a cationic polymer (e.g., 0.1-2%, or even 0.2-0.8%) to provide improved appearance, feel or deposition benefits to hair or skin. The cationic polymer can have a weight average molecular weight of 50 kDa to about 5 MDa (e.g., 500 kDa-4 MDa, 1-3 MDa, 1.2-2 MDa, or even 1.4-1.8 MDa) and a charge density of 0.2 meq/g to 12 meq/g (e.g., 0.4-10 meq/g, 0.4-5 meq/g, 0.4-4 meq/g, 0.4-3 meq/g, or even 0.4-2 meq/g). The charge densities can be measured at the pH of intended use of the personal care composition, which can be pH 3 to pH 9 (e.g., pH 4-8 or pH 4.5-6.5).

The cationic polymers may include cationic, nitrogen-containing moieties such as quaternary ammonium or cationic protonated amino moieties. The cationic protonated amines can be primary, secondary, or tertiary amines, depending upon the particular species and the selected pH of the composition. Anionic counterions can be used in association with the cationic polymers, as long as the polymers remain soluble. Examples of suitable counterions include halide counterions (e.g., chloride, fluoride, bromide, iodide).

Some nonlimiting examples of cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone. Some nonlimiting examples of cationic protonated amino and quaternary ammonium monomers include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Additional nonlimiting examples of cationic polymers include copolymers of 1-vinyl-2-pyrrolidone and 1-vinyl-3-methylimidazolium salt (e.g., chloride salt) (referred to in the industry by the Personal Care Products Council (“PCPC”) as Polyquaternium-16); copolymers of 1-vinyl-2-pyrrolidone and dimethylaminoethyl methacrylate (Polyquaternium-11); cationic diallyl quaternary ammonium-containing polymers, including, for example, dimethyldiallylammonium chloride homopolymer, copolymers of acrylamide and dimethyldiallylammonium chloride (Polyquaternium-6 and Polyquaternium-7, respectively); amphoteric copolymers of acrylic acid including copolymers of acrylic acid and dimethyldiallylammonium chloride (Polyquaternium-22), terpolymers of acrylic acid with dimethyldiallylammonium chloride and acrylamide (Polyquaternium-39), and terpolymers of acrylic acid with methacrylamidopropyl trimethylammonium chloride and methylacrylate (Polyquaternium-47). In some aspects, suitable cationic substituted monomers include cationic substituted dialkylaminoalkyl acrylamides, dialkylaminoalkyl methacrylamides, and combinations thereof. The cationic polymer can be AM:TRIQUAT which is a copolymer of acrylamide and 1,3-Propanediaminium, N-[2-[[[dimethyl[3-[(2-methyl-1-oxo-2-propenyl)amino]propyl]ammonio]acetyl]amino]ethyl]2-hydroxy-N,N,N′,N′,N′-pentamethyl-, trichloride (Polyquaternium-76). AM: TRIQUAT may have a charge density of 1.6 meq/g and a molecular weight of 1.1 MDa.

In some aspects, the cationic monomer can be polymethyacrylamidopropyl trimonium chloride, available under the trade name Polycare® 133, from Solvay (Brussels, Belgium). Copolymers of the cationic monomer may also suitable, and the charge density of the total copolymer can be 2.0 meq/g to 4.5 meq/g.

Other cationic polymers include polysaccharide polymers, such as cationic cellulose derivatives and cationic starch derivatives. In certain embodiments, a cationic cellulose polymer can be selected from the salts of hydroxyethyl cellulose reacted with trimethyl ammonium substituted epoxide, referred to in the industry (PCPC) as Polyquaternium-10 and available from Dow Chemical Company as UCARE™ JR-30M, KG-30 M and LR-30M. Other examples of cationic cellulose polymers include polymeric quaternary ammonium salts of hydroxyethyl cellulose reacted with lauryl dimethyl ammonium-substituted epoxide referred to in the industry (PCPC) as Polyquaternium-24.

Further examples of cationic polymers include cationic guar gum derivatives, such as guar hydroxypropyltrimonium chloride, such as the Jaguar® series available from Solvay and the N-Hance™ and AquaCat™ series from Ashland (Wilmington, Delaware). Additional disclosure of cationic guar gum derivatives can be found in U.S. Pat. No. 6,930,078.

In some instances, the cationic polymer may include a synthetic cationic polymer or derivative thereof present at 0.025% to about 5%. Preferred synthetic cationic polymers are generally water-soluble or dispersible and non-crosslinked. In some instances, the synthetic cationic polymer can be a copolymer that includes one or more cationic monomer units and one or more nonionic or anionic monomer units, as long as the copolymer has a net positive charge. Synthetic cationic polymers can have a cationic charge density of 0.5 meq/g to 12 meg/g and an average molecular weight of 1 kDa to 5 MDa. Some non-limiting examples of synthetic cationic polymers are described in US 2003/0223951.

### Carrier

The composition may optionally include 20-95% of an aqueous carrier such as water and/or a water miscible solvent. The type and amount of aqueous carrier should be selected to provide the composition with the desired rheological properties. The liquid carrier can be water with, e.g., less than 10%, 7%, 5%, 3%, 1%, 0.5% or even 0% miscible organic solvent. Some nonlimiting examples of organic solvents include lower alkyl alcohols (e.g., ethanol and isopropanol) and polyhydric alcohols (e.g., propylene glycol, hexylene glycol, glycerin, and propane diol).

### Optional Ingredients

The personal care compositions described herein may include a variety of optional ingredients to tailor the properties and characteristics of the composition, as desired. The optional ingredients may be materials that are commonly included in compositions of the type. The optional ingredients should be physically and chemically compatible with the essential components of the personal care composition and should not otherwise unduly impair the stability, aesthetics, or performance of the composition. Individual concentrations of optional components can generally range from 0.001% to 10%.

Some non-limiting examples of optional ingredients that can be included in the personal care compositions herein include deposition aids, cationic polymers, conditioning agents (including gel network, triglyceride oils, hydrocarbon oils, fatty esters, silicones), anti-dandruff agents (e.g., zinc pyrithione, zinc carbonate, piroctone olamine, piroctone, ciclopirox, rilopirox, MEA-Hydroxyoctyloxypyridinone, azoxystrobin, sulfur, azoles, salicylic acid and selenium sulfide, 1,10-phenanthroline), anti-microbial agents, suspending agents, viscosity modifiers, dyes, pigments, nonvolatile solvents or diluents (water soluble and insoluble), pearlescent aids, foam boosters, pediculocides, pH adjusting agents, perfumes, preservatives, chelants, proteins, vitamins, amino acids, skin active agents, sunscreens, UV absorbers, stabilizers, and combinations of these.

### Method of Making a Personal Care Composition

The personal care composition described herein can be made using conventional methods for making compositions of the type desired (e.g., shampoo, conditioner or body wash). A particularly suitable method of making the compositions herein is described in Example 1 below. In some aspects, the composition may include a gel network to aid in the conditioning of hair or scalp. U.S. Publication No. 2006/269501 discloses methods of making gel networks that may be suitable for use herein.

### Method of Use

The personal care compositions described herein can be used in a conventional manner for cleansing and conditioning of hair or skin. Effective amounts of the composition for use generally range from 1 g to 50 g (e.g., 1 g to about 20 g). Generally, a method of treating hair or skin can include applying the personal care composition to the hair or skin. For example, an effective amount of the personal care composition can be applied to the hair or skin, which has been wetted with water, and then the composition can be rinsed off. Application to the hair typically includes working the composition through the hair such that most or all of the hair is contacted with the composition. The personal care composition can be used as a liquid, solid, semi-solid, flake, gel, foam, in a pressurized container with a propellant added, or used in a pump spray form. The viscosity of the product may be selected to accommodate the form desired.

In some aspects, the method for treating the hair or skin can include the steps of: (a) wetting the hair or skin with water; (b) applying an effective amount of the personal care composition to the hair or skin, and (c) rinsing the applied areas of skin or hair with water. These steps can be repeated as many times as desired to achieve the desired cleansing and conditioning benefit.

### Methods

### Blender Lather Height

Consumers commonly associate foaming and lathering with the quality of a personal cleansing composition such as a shampoo. This method provides a way to simulate the lather produced by surfactants when used on hair under typical shampooing conditions and quantify certain lather properties. Oil (e.g., sebum) is one of the most common contaminates found on hair that can undesirably affect the lather properties of a shampoo. Thus, this method evaluates the effect of oil on lather properties.

100 mL of tap water, which has a water hardness of about 6-8 gpg (at 100° F.), is added to a suitable blender (e.g., KitchenAid KSB560CUI brand food mixer or equivalent), followed by 2 mL of the test composition and 1 mL of extra virgin olive oil. Blend the mixture on “stir” setting for 30 seconds and measure the height of the lather within the blender in centimeters and record as Lather Height.

In some instances, it may be desirable to calculate the Lather Height to be expected from a mixture of acyl taurate and N-alkyl acyl taurate, based on the weight average Lather Height of the individual surfactants. At a constant co-surfactant concentration, the weighted average Lather Height of a composition containing a mixture of acyl taurate and N-alkyl acyl taurate can be calculated using the following equation:

$$weighted average Lather Height = \frac{{w}_{a} {X}_{a} + {w}_{b} {X}_{b}}{{w}_{a} + {w}_{b}}$$

Where Wa=wt % of acyl taurate surfactant in taurate mixture composition
Xa=Lather Height of acyl taurate only composition
Wp=wt % of N-alkyl acyl taurate surfactant in taurate mixture composition
Xa=Lather Height of N-alkyl acyl taurate only composition

### Rheology Method

Personal care composition viscosities can be measured on a 2.5 mL sample using a cone and plate Brookfield® RS brand rheometer with cone C75-1 at 2 s⁻¹, 27° C. at 3 mins.

### Solubility Method

This method can be used to determine the solubility characteristics of a material (e.g., an acyl taurate) in a composition. The method uses heating and cooling cycles to simulate environmental conditions commonly experienced by a personal care product and accelerate the processes that can lead to product instability.

The test composition (e.g., an aqueous surfactant composition consisting of an acyl taurate surfactant and an N-alkyl acyl taurate surfactant and/or a co-surfactant, and/or a cationic polymer and water) is placed in a closed container to prevent evaporation and heated at 40° C. for a minimum of 30 minutes or until the composition is visually clear & free of precipitates. One mL of the test solution is added to a Crystal-16 vial, closed with a basic cap and placed into a Technobis Crystallization Systems Crystal16® Model 2.2 or equivalent. The % transmittance readings are set to record 1 measurement every 300 seconds. The vials are cooled to 5° C. at a rate of 0.25° C./min and held at 5° C. for 8 hours (cooling cycle). The vials are then heated to 20° C. at a rate of 0.25° C./min and held at 20° C. for 8 hours (heating cycle). After the cooling and heating cycles, the vials are removed from the machine and visually checked for any precipitate. When the final % T recorded at the end of the heating cycle is greater than or equal to 90% and no precipitate is observed the sample is considered soluble.

### EXAMPLES

### Example 1: Example Formulations

Table 1 provides prophetic examples of inventive personal care composition formulations. The compositions in Table 1 can be made by adding DI water to a mixing vessel and then adding each subsequent ingredient while stirring. Surfactants with low water solubility such as cocamide MEA or sodium cocoyl isethionate, if present, require the composition to be heated to 50-75° C. and stirred until fully solubilized (i.e., no visible particles remain and batch is clear). The remaining ingredients, except for cationic polymer or volatile materials, are then added to the mixing vessel and mixed until fully dissolved or solubilized. If heated, the composition is then cooled to 35° C. or less before volatile ingredients such as perfume are added. If cationic polymer is present, in a separate container, the cationic polymer is mixed with water at a 1:20 ratio (polymer:water) to form a slurry or dilute solution, which is then added to the cooled composition in the mixing vessel and mixed for 10 minutes. The pH of the composition is adjusted with citric acid (typically, 0.2-0.5%). Viscosity is adjusted with sodium chloride. DI water is added to bring the final volume to 100%. The mixture is mixed until homogeneous (˜10 minutes).

TABLE 1

| | 1 | 2 | 3 | 4 | 5 | 6 | 7 | 8 |
|---|---|---|---|---|---|---|---|---|
| | Active Wt % | | | | | | | |
| SLT ¹ | 9.0 | 6.3 | 3.2 | 6.3 | 7.2 | 4.5 | 1.8 | 2.0 |
| SMLT ² | 1.0 | 0.7 | 0.8 | 2.7 | 4.8 | 4.5 | 4.2 | 8.0 |
| Ratio of SLT to SMLT on | 90:10 | 90:10 | 80:20 | 70:30 | 60:40 | 50:50 | 30:70 | 20:80 |
| 100% taurate basis | | | | | | | | |
| Sodium C14-16 Olefin | | 7 | 5 | | 2 | | 6.5 | |
| Sulfonate ³ | | | | | | | | |
| Coco-betaine ⁴ | 6 | 2 | | | 3 | 5 | | |
| Cocamide MEA ⁵ | | | | 0.8 | 1 | | | |
| Decyl Glucoside ⁶ | | | 1 | 2 | | | | 10 |
| Guar | 0.25 | | | | 1 | | 0.1 | |
| Hydroxypropyltrimonium | | | | | | | | |
| Chloride ⁷ | | | | | | | | |
| Guar | | | | 0.6 | | | | |
| Hydroxypropyltrimonium | | | | | | | | |
| Chloride ⁸ | | | | | | | | |
| Guar | | 0.4 | 0.25 | | | | | |
| Hydroxypropyltrimonium | | | | | | | | |
| Chloride ⁹ | | | | | | | | |
| AM:Triquat ¹⁰ | | | 0.08 | | | | | |
| Polyquaternium-6 ¹¹ | 0.1 | | | | | 0.25 | | |
| Piroctone Olamine ¹² | 1 | | 0.25 | | | 0.5 | | |
| Dimethiconol ¹³ | | 0.5 | | | 1 | | 0.2 | |
| Tetrasodium EDTA | 0.13 | 0.13 | 0.13 | 0.13 | 0.13 | 0.13 | 0.13 | 0.13 |
| Sodium Benzoate | 0.25 | 0.25 | 0.15 | | 0.75 | 0.15 | 0.2 | 0.25 |
| Sodium Salicylate | 0.25 | 0.25 | 0.15 | | 0.25 | 0.25 | 0.2 | 0.25 |
| Methylchloroisothiazolinone/ | | | | 5 ppm | | | | |
| Methylisothiazolinone ¹⁴ | | | | | | | | |
| Perfume | 0.5 | 1.2 | 1 | 1 | 1 | 0.8 | 1 | 1 |
| Citric Acid | to | to | to | to | to | to | to | to |
| | pH 4.5 | pH 5.0 | pH 5.3 | pH 6.5 | pH 6.0 | pH 5.7 | pH 5.2 | pH 5.5 |
| Sodium Chloride | 0 | 1 | 1 | 0.9 | 0.5 | 2 | 0.2 | 0 |
| (excludes carryover) | | | | | | | | |
| Water | QS | QS | QS | QS | QS | QS | QS | QS |

¹ Sodium Lauroyl Taurate from P&G Chemicals
² Sodium Methyl Lauroyl Taurate from P&G Chemicals or Geropon ® TL 32 L from Solvay
³ Bio-terge ® AS-40 HP from Stepan
⁴ Dehyton ® AB 30 from BASF
⁵ Comperlan ® CMEA from BASF
⁶ Plantaren ® 2000 N UP from BASF
⁷ Jaguar ® C-500 (MW = 500 kDA, CD = 0.8 meq/g) from Solvay
⁸ N-Hance ™ 3000 (MW = 1.7 MDa, CD = 0.4 meq/g) from Ashland
⁹ N-Hance ™ 3196 (MW = 2 MDa, CD = 0.8 meq/g) from Ashland
¹⁰ Mirapol ® AT 1 from Solvay
¹¹ Flocare ™ C 106 MSS from SNF Inc.
¹² Octopirox ® from Clariant
¹³ Xiameter ™ MEM-1872 Emulsion from Dow
¹⁴ Kathon ™ broad-spectrum microbicide

### Example 2: Solubility Improvement

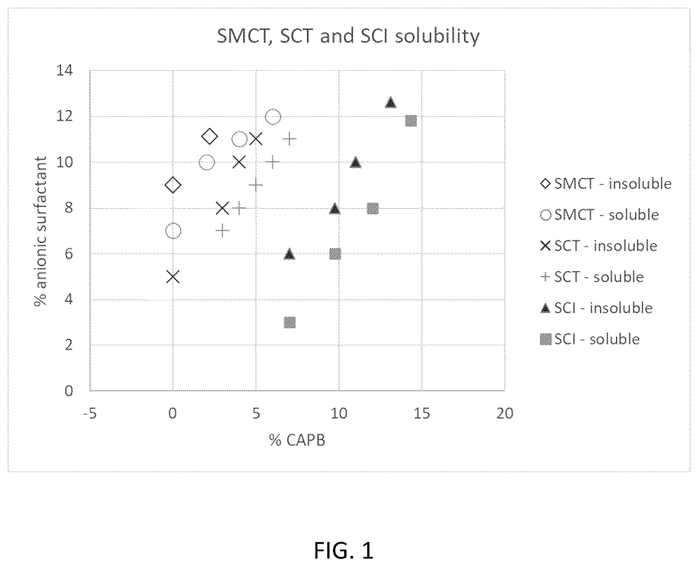

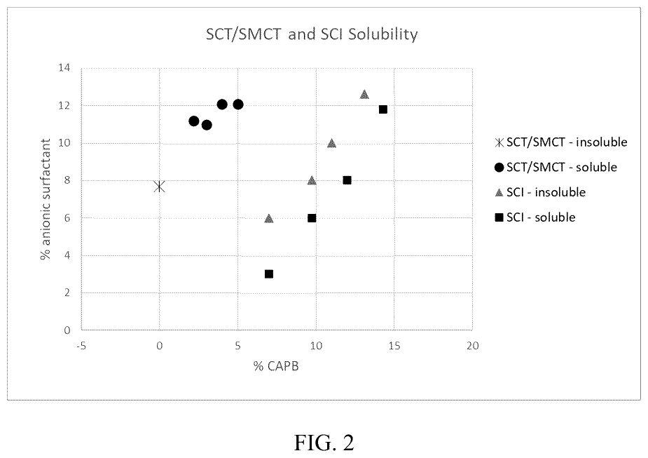

This example demonstrates the unexpected solubility benefit provided by a combination of an acyl taurate and an N-alkyl acyl taurate. The anionic surfactants used in this example are sodium cocoyl isethionate (SCI), sodium cocoyl taurate (SCT) and sodium methyl cocoyl taurate (SMCT). The amphoteric co-surfactant is cocamidopropyl betaine (CAPB). The test compositions for each leg of this example are prepared as aqueous solutions, as shown in Tables 2A to 2E. The test results are summarized in Tables 2B to 2E and illustrated in FIGS. 1 and 2.

TABLE 2A
Base formulation

| | Solubility Compositions |
|---|---|
| | Active Wt % |
| CAPB ¹ | 0-14.3 (see tables below for exact levels) |
| Anionic surfactants | 3.0-12.6 (see tables below for exact levels) |
| Methylchloroisothiazolinone/ | 0.0005 |
| Methylisothiazolinone ⁶ | |
| Citric Acid | adjustable to reach pH 5.2 |
| Water | QS |

TABLE 2B
SCI

| | SCI Solubility Compositions | | | | | | | |
|---|---|---|---|---|---|---|---|---|
| | SCI-1 | SCI-2 | SCI-3 | SCI-4 | SCI-5 | SCI-6 | SCI-7 | SCI-8 |
| | Active Wt % | | | | | | | |
| CAPB ¹ | 7 | 7 | 9.75 | 9.75 | 12 | 11 | 14.3 | 13.1 |
| SCI ² | 3 | 6 | 6 | 8 | 8 | 10 | 11.8 | 12.6 |
| Soluble | yes | no | yes | no | yes | no | yes | no |

TABLE 2C
SCT

| | SCT Solubility Compositions | | | | | | | | |
|---|---|---|---|---|---|---|---|---|---|
| | SCT-1 | SCT-2 | SCT-3 | SCT-4 | SCT-5 | SCT-6 | SCT-7 | SCT-8 | SCT-9 |
| | Active Wt % | | | | | | | | |
| CAPB ¹ | 0 | 3 | 3 | 4 | 4 | 5 | 5 | 6 | 7 |
| SCT ³ | 5 | 7 | 8 | 8 | 10 | 9 | 11 | 10 | 11 |
| Soluble | no | yes | no | yes | no | yes | no | yes | yes |

TABLE 2D
SMCT

| | SMCT Solubility Compositions | | | | | |
|---|---|---|---|---|---|---|
| | SMCT-1 | SMCT-2 | SMCT-3 | SMCT-4 | SMCT-5 | SMCT-6 |
| | Active Wt % | | | | | |
| CAPB ¹ | 0 | 0 | 2 | 2.2 | 4 | 6 |
| SMCT ⁴ | 7 | 9 | 10 | 11.1 | 11 | 12 |
| Soluble | yes | no | yes | no | yes | yes |

TABLE 2E
SCT + SMCT

| | SCT/SMCT Mixture Solubility Compositions | | | | |
|---|---|---|---|---|---|
| | Mix-1 | Mix-2 | Mix-3 | Mix-4 | Mix-5 |
| | Active Wt % | | | | |
| CAPB ¹ | 0 | 3 | 4 | 5 | 2.2 |
| SCT ³ | 7 | 10 | 11 | 11 | 10.2 |
| SMCT ⁴ | 0.7 | 1.0 | 1.1 | 1.1 | 1.0 |
| Total anionic surfactant | 7.7 | 11.0 | 12.1 | 12.1 | 11.2 |
| (SCT + SMCT) | | | | | |
| Soluble | no | yes | yes | yes | yes |

¹ Tego ® Betain CK pH 12 from Evonik
2. Hostapon ® SCI 85 C from Clariant
³ Sodium Cocoyl Taurate from P&G Chemicals
⁴ Sodium Methyl Cocoyl Taurate from P&G Chemicals or Pureact ® WS Conc from Innospec
5. Kathon ™ broad-spectrum microbicide

FIG. 1 illustrates the solubility of SCT, SMCT and SCI. As expected, SCT exhibited better solubility than SCI, but poorer solubility than SMCT. Thus, more amphoteric co-surfactant (cocamidopropyl betaine) is required to solubilize the less soluble anionic surfactant, which can be undesirable.

FIG. 2 illustrates the solubility of a combination of SCT (91%) and SMCT (9%) versus SCI. As can be seen in FIG. 2, combinations of SCT and SMCT are soluble at concentrations where a lower level of SCT alone would be insoluble. For example, composition Mix-2 (3% CAPB+10% SCT+1% SMCT) is soluble, whereas composition SCT-3 (3% CAPB+8% SCT) is not. This is unexpected because composition Mix-2 contains 25% more SCT than composition SCT-3 (i.e., 10% versus 8%), and thus one would not expect a higher amount of SCT to be soluble with the same amount of co-surfactant (i.e., 3%).

Perhaps more surprising is that a combination of SCT and SMCT was found to be soluble at concentrations where SMCT is insoluble. This is surprising because SMCT is generally more soluble than SCT. For example, composition Mix-5 (2.2% CAPB+10.2% SCT+1% SMCT) is soluble, whereas composition SMCT-4 (2.2% CAPB+11.1% SMCT) is not. Generally, while one might expect the solubility of the combination of SCT and SMCT to be greater than that of SCT alone, one would not expect the combination to be more soluble than SMCT alone, especially considering the amount of SMCT present is only a very small fraction of the total surfactant in composition Mix-5.

### Example 3

This example demonstrates that even small changes in N-alkyl acyl taurate level can have a meaningful impact on acyl taurate solubility. Table 3 compares the solubility of sodium lauroyl taurate at three different levels (8%, 9% and 11%) in a composition comprising sodium methyl lauroyl taurate. The weight ratio of SLT to SMLT ranges from 10.5:1 to 8:1. The co-surfactant, cocamidopropyl betaine (CAPB), is kept constant in each test leg. As can be seen in Table 3, changes in SMLT level as low as 0.08% SMLT can be the difference between SLT solubility and insolubility.

TABLE 3

| | Test leg 1 | | Test leg 2 | | Test leg 3 | |
|---|---|---|---|---|---|---|
| Ingredient | Active wt % | | | | | |
| SLT | 8 | 8 | 9 | 9 | 11 | 11 |
| SMLT | 0.77 | 0.85 | 0.95 | 1.13 | 1.05 | 1.17 |
| CAPB | 2 | 2 | 3 | 3 | 5 | 5 |
| Methylchloroisothiazolinone/ | 0.0005 | 0.0005 | 0.0005 | 0.0005 | 0.0005 | 0.0005 |
| Methylisothiazolinone | | | | | | |
| Citric Acid | to | to | to | to | to | to |
| | pH 5.2 | pH 5.2 | pH 5.2 | pH 5.2 | pH 5.2 | pH 5.2 |
| Water | QS | QS | QS | QS | QS | OS |
| Ratio of SLT to SMLT | 10.5:1 | 9.4:1 | 9.4:1 | 8:1 | 10.5:1 | 9.4:1 |
| Soluble | No | Yes | No | Yes | No | Yes |

### Example 4: Effect of Co-Surfactant on Acyl Taurate Solubility

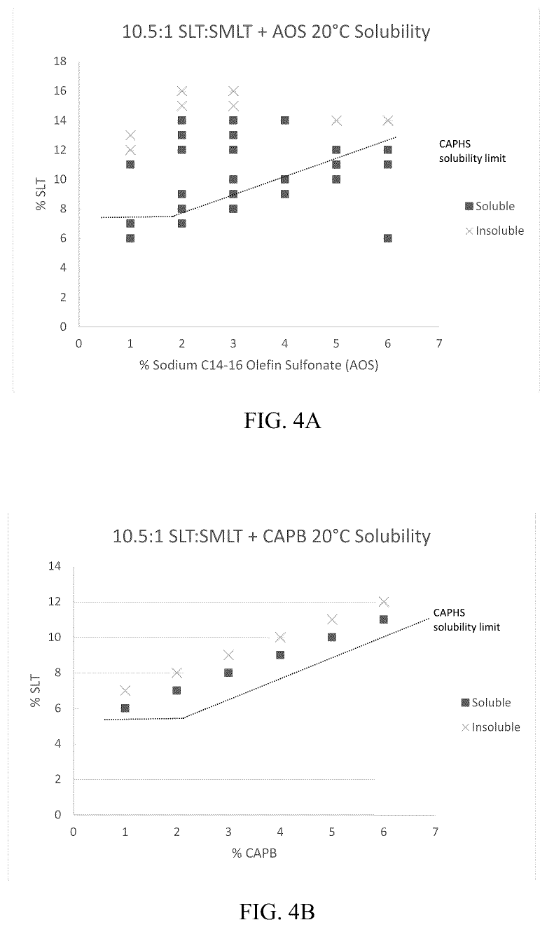

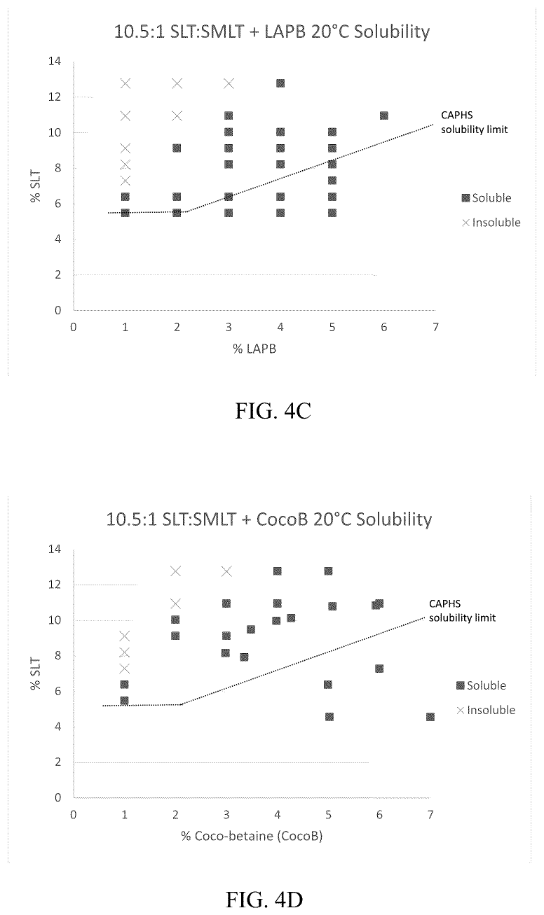

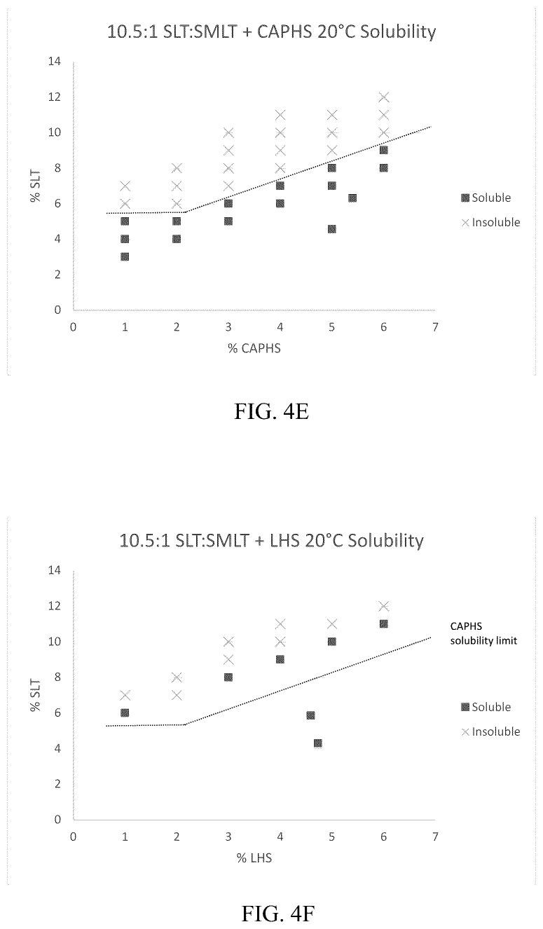

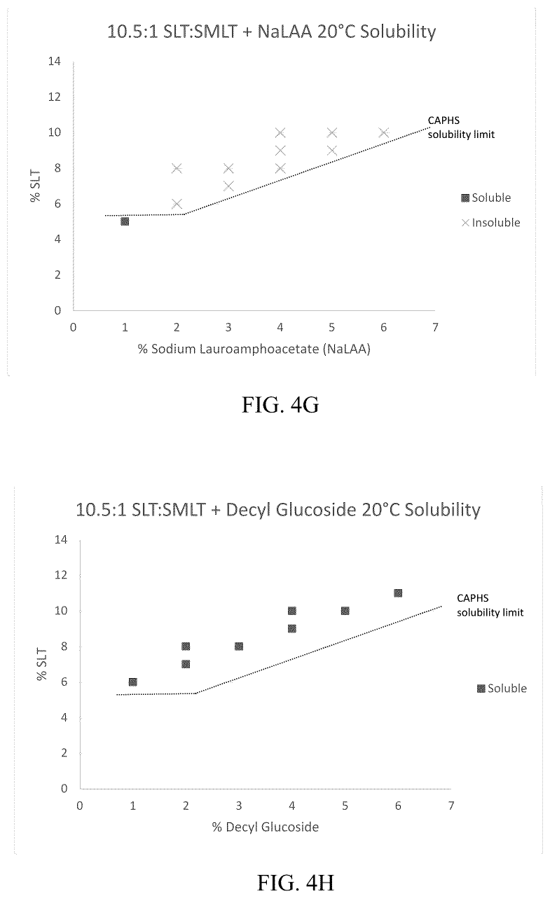

The example demonstrates how selection of a co-surfactant can affect acyl taurate solubility in a personal care composition. The test compositions for each leg of this example are prepared as aqueous solutions, as shown in Table 4. The test results are summarized in Table 4A and 4B and illustrated in FIG. 4A-4H. Unless indicated otherwise, the materials used in these examples can be sourced from the same suppliers as described in any of the previous Examples.

TABLE 4A

| | A | | B | | C | | D | |
|---|---|---|---|---|---|---|---|---|
| Ingredient | Active Wt % | | | | | | | |
| SLT | 6 | 9 | 6.38 | 9.12 | 6 | 9 | 6.38 | 9.47 |
| SMLT | 0.57 | 0.86 | 0.61 | 0.87 | 0.57 | 0.86 | 0.61 | 0.91 |
| Sodium C14-16 Olefin | 1 | 4 | | | | | | |
| Sulfonate (AOS) | | | | | | | | |
| Cocamidopropyl | | | 1 | 4 | | | | |
| Betaine (CAPB) | | | | | | | | |
| Lauramidopropyl | | | | | 1 | 4 | | |
| Betaine (LAPB) | | | | | | | | |
| Coco-Betaine (CocoB) | | | | | | | 1 | 3.48 |
| Methylchloroisothiazolinone/ | 5 ppm | 5 ppm | 5 ppm | 5 ppm | 5 ppm | 5 ppm | 5 ppm | 5 ppm |
| Methylisothiazolinone | | | | | | | | |
| Citric Acid | to | to | to | to | to | to | to | to |
| | pH 5.2 | pH 5.2 | pH 5.2 | pH 5.2 | pH 5.2 | pH 5.2 | pH 5.2 | pH 5.2 |
| Water | QS | QS | QS | QS | QS | QS | QS | QS |
| Total Taurate surfactants | 6.6 | 9.9 | 7.0 | 10.0 | 6.6 | 9.9 | 7.0 | 10.4 |
| Ratio of SLT to SMLT | 10.5:1 | 10.5:1 | 10.5:1 | 10.5:1 | 10.5:1 | 10.5:1 | 10.5:1 | 10.5:1 |
| Soluble | Yes | Yes | Yes | Yes | Yes | Yes | Yes | Yes |

TABLE 4B

| | E | | F | | G | | H | |
|---|---|---|---|---|---|---|---|---|
| Ingredient | Active Wt % | | | | | | | |
| SLT | 6 | 9 | 6 | 9 | 6 | 9 | 6 | 9 |
| SMLT | 0.57 | 0.86 | 0.57 | 0.86 | 0.57 | 0.86 | 0.57 | 0.86 |
| Cocamidopropyl | 1 | 4 | | | | | | |
| Hydroxysultaine (CAPHS) ¹ | | | | | | | | |
| Lauryl Hydroxysultaine (LHS) ² | | | 1 | 4 | | | | |
| Sodium Lauroamphoacetate | | | | | 1 | 4 | | |
| (NaLAA) ³ | | | | | | | | |
| Decyl Glucoside | | | | | | | 1 | 4 |
| Methylchloroisothiazolinone/ | 5 ppm | 5 ppm | 5 ppm | 5 ppm | 5 ppm | 5 ppm | 5 ppm | 5 ppm |
| Methylisothiazolinone | | | | | | | | |
| Citric Acid | to | to | to | to | to | to | to | to |
| | pH 5.2 | pH 5.2 | pH 5.2 | pH 5.2 | pH 5.2 | pH 5.2 | pH 5.2 | pH 5.2 |
| Water | QS | QS | QS | QS | QS | QS | QS | QS |
| Total Taurate surfactants | 6.6 | 9.9 | 6.6 | 9.9 | 6.6 | 9.9 | 6.6 | 9.9 |
| Ratio of SLT to SMLT | 10.5:1 | 10.5:1 | 10.5:1 | 10.5:1 | 10.5:1 | 10.5:1 | 10.5:1 | 10.5:1 |
| Soluble | No | No | Yes | Yes | No | No | Yes | Yes |

¹ StarSurf ™ CAPHS from StarChem
² Mirataine ® LHS from Syensqo
³ Miranol ® Ultra L-32 MB from Syensqo

As can be seen in Tables 4A and 4B and FIGS. 4A-4H, it can be important to select an appropriate co-surfactant to ensure adequate acyl taurate solubility. It is generally desirable to minimize the amount of co-surfactant added to the composition in order to build viscosity, but it is also desirable to have the formulation flexibility to add an appropriate amount of co-surfactant (e.g., 0.5%-10% or 1%-5%).

In this example, the zwitterionic surfactant cocamidopropyl hydroxysultaine (CAPHS) and the amphoteric surfactant sodium lauroamphoacetate did not provide the desired solubility. This result is unexpected considering the suitability of other amphoteric/zwitterionic surfactants. The results are particularly unexpected for CAPHS, due to the chemical/structural similarity of CAPHS to lauryl hydroxysultaine (LHS) and cocamidopropyl betaine (CAPB). The solubility line for CAPHS is included in FIGS. 4A-4H to provide context for the solubility effects of the other co-surfactants.

### Example 5: Comparative Example

This example demonstrates the inability of certain cosurfactants to provide the desired viscosity building in a personal care composition. Application Examples A1 and A5 from China Publication No. CN116098821 were reproduced as shown in Table 5. The ingredient amounts shown in Table 5 are on a 100% active basis and reflect the active level calculated by multiplying the ingredient level by the ingredient activity indicated as the raw material specification in CN116098821. The CN116098821 publication indicates that PEG-150 stearate was used in these examples. However, PEG-150 stearate was not readily available, and so the example formulations were made with PEG-100 stearate and PEG-150 distearate, instead. The molecular structures of PEG-100 stearate and PEG-150 distearate are such that their impact on composition viscosity should bracket the viscosity impact of PEG-150 stearate. In other words, the viscosity of PEG-150 stearate should fall between the viscosities of PEG-100 stearate and PEG-150 distearate shown in Table 5. The viscosity of the composition was measured according to the Rheology method described herein above. The rheometer when measuring at the shear rate and with the cone indicated in the Rheology method has a lower detection limit of about 750 mPa-s. Thus, viscosities below this amount are indicated as <750.

TABLE 5

| | A1 | A5 | A1-100 | A5-100 |
|---|---|---|---|---|
| Ingredient | Active wt % | | | |
| SLT | 7.695 | 0.855 | 7.695 | 0.855 |
| SMLT | 0.855 | 7.695 | 0.855 | 7.695 |
| Cocamidopropyl | 6.6 | 6.6 | 6.6 | 6.6 |
| hydroxysultaine (CAPHS) ¹ | | | | |
| Polyquaternium-7 ² | 0.03 | 0.03 | 0.03 | 0.03 |
| PEG-150 distearate ³ | 0.2 | 0.2 | | |
| PEG-100 stearate ⁴ | | | 0.2 | 0.2 |
| Citric acid | To pH 5.5 | | | |
| Water | QS | | | |
| Ratio of SLT:SMLT | 9:1 | 1:9 | 9:1 | 1:9 |
| Viscosity (mPa-s) | 862 | <750 | 1051 | <750 |

¹ StarSurfTM CAPHS from StarChem.
² Merquat ™ 550 PR Polymer from Lubrizol
³ Hallstar ® PEG 6000 DS from Hallstar
⁴ Hallstar ® PEG 4400 MS MB from Hallstar

As can be seen in Table 5, the compositions do not have a suitable viscosity, which further emphasizes the importance of selecting the appropriate combination of acyl taurate, N-alkyl acyl taurate and cosurfactant.

### Example 6-Lather Benefit

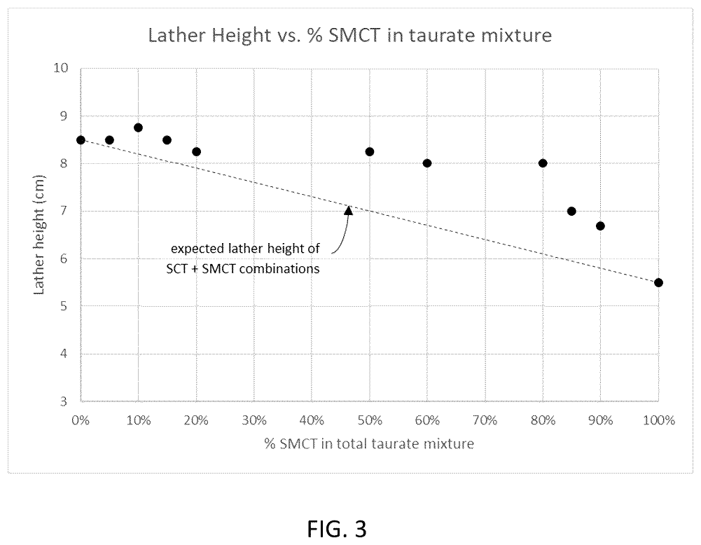

This example demonstrates the ability of the inventive SCT/SMCT surfactant system to provide good foaming and lather. Lather is an important property of a personal care composition such as a shampoo or body wash because consumers generally associate lather properties (e.g., amount and creaminess) with product performance and quality. The compositions tested in this Example were prepared as described above and tested according to the Blender Lather Height method. The results of the test are summarized below in Table 6 and illustrated in FIG. 3. Inventive compositions are designated as “Inv.” and comparative compositions are designated as “C.” A Lather Height of more than 7.5 cm is desirable, a Lather Height of 6-7.5 cm may be acceptable in some instances, but is generally not preferred, and a Lather Height of less than 6 cm is undesirable.

TABLE 6

| | 7 (C) | 8 (Inv) | 9 (C) | 10 (C) | 11 (Inv) | 12 (Inv) | 13 (Inv) | 14 (Inv) | 15 (Inv) |
|---|---|---|---|---|---|---|---|---|---|
| | Active wt % | | | | | | | | |
| SCT ¹ | 9 | 8.2 | | 9 | 8.55 | 8.2 | 7.65 | 7.2 | 4.5 |
| SMCT ² | | 0.8 | 9 | | 0.45 | 0.8 | 1.35 | 1.8 | 4.5 |
| Lauramidopropyl betaine ⁶ | 7 | 7 | 7 | 7 | 7 | 7 | 7 | 7 | 7 |
| Polyquaternium-10 ⁷ | 0 | 0 | 0 | 0.5 | 0.5 | 0.5 | 0.5 | 0.5 | 0.5 |
| Tetrasodium EDTA | 0.13 | 0.13 | 0.13 | 0.13 | 0.13 | 0.13 | 0.13 | 0.13 | 0.13 |
| Sodium Benzoate | 0.25 | 0.25 | 0.25 | 0.25 | 0.25 | 0.25 | 0.25 | 0.25 | 0.25 |
| Sodium Salicylate | 0.25 | 0.25 | 0.25 | 0.25 | 0.25 | 0.25 | 0.25 | 0.25 | 0.25 |
| Perfume | 1 | 1 | 1 | 1 | 1 | 1 | 1 | 1 | 1 |
| Sodium Chloride | 1.5 | 1.5 | 1.5 | 0.8 | 0.8 | 0.8 | 0.8 | 0.8 | 0.8 |
| (excludes carryover) | | | | | | | | | |
| Citric Acid | To | To | To | To | To | To | To | To | To |
| | pH 5.6 | pH 5.5 | pH 5.5 | pH 5.6 | pH 5.5 | pH 5.5 | pH 5.6 | pH 5.6 | pH 5.5 |
| Water | QS | QS | QS | QS | QS | QS | QS | QS | QS |
| Ratio of acyl taurate to N- | | 91:9 | | | 95:5 | 91:9 | 85:15 | 80:20 | 50:50 |
| alkyl acyl taurate (on a | | | | | | | | | |
| 100% taurate basis) | | | | | | | | | |
| Lather height (cm) | 8.3 | 9 | 7.5 | 8.5 | 8.5 | 8.75 | 8.5 | 8.25 | 8.25 |

| | 16 (Inv) | 17 (Inv) | 18 (Inv) | 19 (Inv) | 20 (C) | 21 (Inv) | 22 (C) | 23 (C) |
|---|---|---|---|---|---|---|---|---|
| | Active wt % | | | | | | | |
| SCT ¹ | 3.6 | 1.8 | 1.35 | 0.8 | | 5.5 | | |
| SMCT ² | 5.4 | 7.2 | 7.65 | 8.1 | 9 | 0.5 | | 6 |
| SCI ⁸ | | | | | | | 6 | |
| Cocamidopropyl betaine ⁵ | | | | | | 4.4 | 4.4 | 4.4 |
| Lauramidopropyl betaine ⁶ | 7 | 7 | 7 | 7 | 7 | 5.4 | 5.4 | 5.4 |
| Polyquaternium-10 ⁷ | 0.5 | 0.5 | 0.5 | 0.5 | 0.5 | 0.25 | 0.25 | 0.25 |
| Tetrasodium EDTA | 0.13 | 0.13 | 0.13 | 0.13 | 0.13 | 0.13 | 0.13 | 0.13 |
| Sodium Benzoate | 0.25 | 0.25 | 0.25 | 0.25 | 0.25 | 0.75 | 0.75 | 0.75 |
| Sodium Salicylate | 0.25 | 0.25 | 0.25 | 0.25 | 0.25 | 0.45 | 0.45 | 0.45 |
| Perfume | 1 | 1 | 1 | 1 | 1 | 1 | 1 | 1 |
| Sodium Chloride | 0.8 | 0.8 | 0.8 | 0.8 | 0.8 | | | |
| (excludes carryover) | | | | | | | | |
| Citric Acid | To | To | To | To | To | To | To | To |
| | pH 5.5 | pH 5.5 | pH 5.5 | pH 5.5 | pH 5.5 | pH 5.5 | pH 5.5 | pH 5.5 |
| Water | QS | QS | QS | QS | QS | QS | QS | QS |
| Ratio of acyl taurate to N- | 40:60 | 20:80 | 15:85 | 10:90 | | 91:9 | | |
| alkyl acyl taurate (on a | | | | | | | | |
| 100% taurate basis) | | | | | | | | |
| Lather height (cm) | 8 | 8 | 7 | 6.7 | 5.5 | 8.3 | 4.5 | 4 |

| | 24 (Inv) | 25 (C) | 26 (Inv) | 27 (Inv) | 28 (Inv) | 29 (Inv) | 30 (Inv) | 31 (Inv) |
|---|---|---|---|---|---|---|---|---|
| | Active wt % | | | | | | | |
| SCT ¹ | 9.9 | | | | | | | |
| SMCT ² | 1.1 | 11 | | | | | | |
| SLT ³ | | | 5.4 | 8.1 | 9.9 | 9.9 | 6.3 | 6.3 |
| SMLT ⁴ | | | 0.6 | 0.9 | 1.1 | 1.1 | 0.7 | 0.7 |
| Cocamidopropyl betaine ⁵ | 5 | 5 | 4.4 | | | | | |
| Lauramidopropyl betaine ⁶ | | | 5.4 | 7 | 5 | 5 | 9 | 9 |
| Polyquaternium-10 ⁷ | | | 0.25 | | | 0.25 | | 0.25 |
| Tetrasodium EDTA | | | 0.13 | 0.13 | 0.13 | 0.13 | 0.13 | 0.13 |
| Methylchloroisothiazolinone/ | 0.0005 | 0.0005 | | | | | | |
| Methylisothiazolinone ⁹ | | | | | | | | |
| Sodium Benzoate | | | 0.75 | 0.25 | 0.25 | 0.25 | 0.25 | 0.25 |
| Sodium Salicylate | | | 0.45 | 0.25 | 0.25 | 0.25 | 0.25 | 0.25 |
| Perfume | | | 1 | 1 | 1 | 1 | 1 | 1 |
| Sodium Chloride | | | | 1 | 1 | 0.9 | 0.5 | 0.15 |
| (excludes carryover) | | | | | | | | |
| Citric Acid | To | To | To | To | To | To | To | To |
| | pH 5.2 | pH 5.2 | pH 5.6 | pH 5.6 | pH 5.5 | pH 5.5 | pH 5.5 | pH 5.5 |
| Water | QS | QS | QS | QS | QS | QS | QS | QS |
| Ratio of acyl taurate to N- | 90:10 | | 90:10 | 90:10 | 90:10 | 90:10 | 90:10 | 90:10 |
| alkyl acyl taurate (on a | | | | | | | | |
| 100% taurate basis) | | | | | | | | |
| Lather height (cm) | 8 | 6.4 | 8.25 | 9 | 8.5 | 8.5 | 8.5 | 8.5 |

¹ Sodium cocoyl taurate from P&G Chemicals
² Sodium methyl cocoyl taurate from P&G Chemicals or Geropon ® TC 95 P or Geropon ® TC 30
³ Sodium lauroyl taurate from P&G Chemicals
⁴ Sodium methyl lauroyl taurate from P&G Chemicals or Geropon ® TL 32 L from Solvay
⁵ Tego ® Betain CK pH 12 from Evonik
⁶ Mirataine ® DAB ULS MB from Syensqo
⁷ UCARE ™ JR30M from Dow
⁸ Hostapon ® SCI 85 C from Clariant
⁹ Kathon ™ broad-spectrum microbicide

As can be seen in Table 6 and FIG. 3, personal care compositions that include a combination of acyl taurate surfactant and N-alkyl acyl taurate surfactant provide better lather than the compositions with a single type of taurate surfactant or an isethionate surfactant. Indeed, the data summarized in Table 6 and illustrated in FIG. 3, show that a combination of acyl taurate and N-alkyl acyl taurate can work synergistically to provide better lather. For example, comparative Compositions 7 (9% SCT) and 9 (9% SMCT) exhibited a lather height of 8.3 and 7.5 cm, respectively. Thus, a combination of SCT and SMCT at the same total amount of anionic surfactant (i.e., 9%) would be expected to provide a Lather Height that is a weighted average of the individual Lather Height values. However, inventive Composition 8 (8.2% SCT+0.8% SMCT) exhibited a Lather Height that is greater than the individual Lather Heights of Compositions 7 and 9 and greater than the weighted average Lather Height of those two compositions, which would be 8.2 cm as calculated below.

$$Where :$$

$${w}_{a} = 8.2 wt \% ,$$

$${X}_{a} = 8.3 cm ,$$

$${w}_{b} = 0.8 wt \% ,$$

$${X}_{b} = 7.5 cm ;$$

$$weighted average Lather Height = \frac{( 8.2 \% ) ( 8.3 cm ) + ( 0.8 \% ) ( 7.5 cm )}{8.2 \% + 0.8 \%} = 8.2 cm$$

Another example of synergy can be seen when comparing Compositions 10 and 20 to Inventive Compositions 11 to 19, which is illustrated in FIG. 3. Comparative Composition 10 (9% SCT) and comparative Composition 20 (9% SMCT) exhibited a Lather Height of 8.5 cm and 5.5 cm, respectively. Thus, a mixture of SCT and SMCT with the same total amount of anionic surfactant would be expected to provide a Lather Height that is a weighted average of the individual Lather Height values. However, as can be seen in Table 6 and in FIG. 3, inventive Compositions 11 and 12 provide the same or greater Lather Height than SCT alone. The Lather Heights exhibited by inventive Compositions 13 to 19, while less than the Lather Height of comparative Composition 10, are still higher than expected, which demonstrates synergy.

The data in Table 6 also suggest that the lather benefit provided by a mixture of SCT and SMCT may be magnified at lower amounts of anionic surfactant (e.g., 6%). For example, comparing inventive Composition 21 to comparative Compositions 22 and 23 appears to show that at 6% anionic surfactant, the combination of SCT and SMCT provide good lather (i.e., Lather Height of 8 or more), whereas the SCI and SMCT exhibit poor lather.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aqueous personal care composition, comprising:
a) an acyl taurate surfactant and an N-alkyl acyl taurate surfactant at a weight ratio of acyl taurate to N-alkyl acyl taurate of about 1:4 to about 50:1;
b) a co-surfactant; and
c) water, wherein the N-alky acyl taurate surfactant and the co-surfactant solubilize the acyl taurate surfactant in the composition, and wherein the composition has a viscosity of about 2000 mPa-s to about 20,000 mPa-s, according to the Rheology method.

2. The personal care composition of claim 1, wherein the weight ratio of acyl taurate to N-alkyl acyl taurate is about 1:1 to about 50:1.

3. The personal care composition of claim 2, wherein the weight ratio of acyl taurate to N-alkyl acyl taurate is about 3:1 to about 50:1.

4. The personal care composition of claim 1, wherein the combination of acyl taurate and N-alkyl acyl taurate exhibits a synergistic Lather Height according to the Blender Lather Height method.

5. The personal care composition of claim 1, wherein the combination of acyl taurate and N-alkyl acyl taurate exhibits synergistic solubility according to the Solubility method.

6. The personal care composition of claim 1, wherein the acyl taurate surfactant is a C8-C24 acyl taurate surfactant.

7. The personal care composition of claim 6, wherein the C8-C24 acyl taurate surfactant is selected from the group consisting of capric ester taurate surfactants, cocoyl taurate surfactants, lauroyl taurate surfactants, myristoyl taurate surfactants, caproyl taurate surfactants, oleoyl taurate surfactants, capryloyl taurate surfactants, palmitoyl taurate surfactants, stearoyl taurate surfactants, linoleoyl taurate surfactants, salts of these and combinations thereof.

8. The personal care composition of claim 1, wherein the N-alkyl acyl taurate surfactant is selected from methyl substituted acyl taurates, ethyl substituted acyl taurates, propyl substituted acyl taurates, butyl substituted acyl taurates, salts of these and combinations thereof.

9. The personal care composition of claim 8, wherein the N-alkyl acyl taurate is selected from the group consisting of methyl capric ester taurate, methyl cocoyl taurate, methyl lauroyl taurate, methyl myristoyl taurate, methyl caproyl taurate, methyl oleoyl taurate, methyl capryloyl taurate, methyl palmitoyl taurate, methyl stearoyl taurate, methyl linoleoyl taurate, salts of these and combinations thereof.

10. The personal care composition of claim 1, wherein the composition further comprises a co-surfactant selected from an alkyl amidopropylbetaine, an alkyl betaine, an alkyl hydroxysultaine, an alpha olefin sulfonate, an alkyl glucoside or a combination thereof.

11. The personal care composition of claim 10, wherein the co-surfactant is selected from cocamidopropyl betaine, lauramidopropyl betaine, coco-betaine, lauryl betaine, lauryl hydroxysultaine, coco-hydroxysultaine, sodium C14-16 olefin sulfonate, sodium C12-14 olefin sulfonate, sodium C12-16 olefin sulfonate, decyl glucoside, lauryl glucoside, coco-glucoside, and combinations thereof.

12. The personal care composition of claim 1, further comprising an additional ingredient selected from deposition aids, conditioning agents, anti-dandruff agents, anti-microbial agents, suspending agents, viscosity modifiers, dyes, pigments, nonvolatile solvents, diluents, pearlescent aids, foam boosters, pH adjusting agents, perfumes, preservatives, chelants, proteins, vitamins, amino acids, skin active agents, sunscreens, UV absorbers, stabilizers, and combinations of these.

13. The personal care composition of claim 12, further comprising an anti-dandruff agent selected from the group consisting of azoles, hydroxyl pyridones, zinc pyrithone, zinc carbonate, piroctone olamine, piroctone, sulfur, ciclopirox, rilopirox, MEA-hydroxyoctyloxypyridinone, salicylic acid, selenium sulfide, azoxystrobin, and 1,10-phenanthroline.

14. The personal care composition of claim 1, wherein the composition is substantially free of sulfated surfactants.

15. A personal care composition, comprising:
a) a surfactant system comprising an acyl taurate surfactant and an N-alkyl acyl taurate surfactant;
b) a cationic polymer; and
c) water.

16. The composition of claim 15, wherein the cationic polymer has at least one of a weight average molecular weight of about 50 kDa to about 5 MDa and a charge density of about 0.2 meq/g to about 12 meq/g.

17. The personal care composition of claim 15, further comprising a co-surfactant selected from cocamidopropyl betaine, lauramidopropyl betaine, coco-betaine, lauryl betaine, lauryl hydroxysultaine, coco-hydroxysultaine, an alpha olefin sulfonate or salt thereof, decyl glucoside, lauryl glucoside, coco-glucoside, and combinations thereof.

18. The composition of claim 15, wherein the N-alkyl acyl taurate surfactant is selected from methyl substituted acyl taurates, ethyl substituted acyl taurates, propyl substituted acyl taurates, butyl substituted acyl taurates, salts of these and combinations thereof.

19. The personal care composition of claim 15, wherein the combination of acyl taurate surfactant and N-alkyl acyl taurate surfactant exhibits synergistic Lather Height according to the Blender Lather Height method.
